(19) United States
(12) Patent Application Publication
(10) Pub. No.: US 2003/0175782 A1
(43) Pub. Date: Sep. 18, 2003

(54) Genetic diagnosis/analysis apparatus and genetic diagnosis support system using the apparatus

(75) Inventors: Kazuhisa Fukushima, Tokyo (JP); Takeo Tanaami, Tokyo (JP)

Correspondence Address: ARMSTRONG,WESTERMAN & HATTORI, LLP, 1725 K STREET, NW, SUITE 1000, WASHINGTON, DC, 20006, US

(73) Assignee: YOKOGAWA ELECTRIC CORPORATION, Tokyo (JP)

(21) Appl. No.: 10/384,706

(22) Filed: Mar. 11, 2003

(30) Foreign Application Priority Data
Mar. 12, 2002 (JP) 2002-067252

(51) Int. Cl. C12Q 1/68; C12M 1/34

(52) U.S. Cl. 435/6; 435/287.2

(57) ABSTRACT

The present invention relates to a genetic diagnosis/analysis apparatus using detection chips for detecting gene expression, gene representation, or the SNPs of genes, from samples of blood, bodily fluids, or affected parts collected from patients, and comprises gene detection means for preprocessing samples and detecting gene expression, gene representation, or the SNPs of genes and an analyzer for analyzing the results of detection by the gene detection means by referring to diagnostic protocols and for obtaining information about the disease in question and other related information.

With such a system configuration as described above, it is possible to perform the preprocessing of the aforementioned samples and gene detection within the same container and the analysis of the resulting data is performed without interruption and completed within an hour.

### BACKGROUND OF THE INVENTION

1. Field of the Invention

The present invention relates to a genetic diagnosis support system using detection chips for detecting gene expression, gene representation, or the single nucleotide polymorphisms (SNPs) of genes, from samples of blood, bodily fluids, or affected parts collected from patients.

2. Description of the Prior Art

Recently, it has become an increasingly common practice to perform diagnosis by detecting genetic abnormalities from DNA or protein using specimens, such as samples of blood, bodily fluids, or affected parts. In this diagnostic method, the fact is that samples of blood, bodily fluids, or affected parts collected from patients are forwarded to a test center and physicians diagnose medical conditions and decide on a treatment policy according to the test results of such specimens.

However, such a conventional diagnosis method as described above usually requires a week or so until the test results provided by the test center become available. Patients must re-visit the hospital to learn the diagnostic results from physicians, thus consuming extra amounts of time.

Another disadvantage is that types of testing are limited and retesting the specimens is not easy. Moreover, such testing is expensive.

It should be noted here that Japanese Laid-open Patent Application 2002-107366 describes a system that assists physicians in making diagnoses by surmising what disease a patient is suffering from currently or will be likely to suffer from in the future, using information obtained from DNA and/or protein.

This system has a control means and a disease-inferring means for inferring related diseases from vast amounts of data obtained from DNA and/or protein. The system also has artificial intelligence functions referred to as a gene knowledge database and a disease-related gene search means. Thus the system is an inference and analysis support system that utilizes diagnosis-related artificial intelligence, in order to obtain better analysis results by constantly updating the content of the database. In conclusion, the system is strictly for the purpose of inferring diseases and is not such as to provide extremely accurate answers to questions regarding the reliability and reproducibility of diagnosis results that are required in a clinic situation.

In addition, this type of system also requires a significant number of days before information is acquired from the DNA and/or protein. Furthermore, patients must re-visit the hospital to learn the diagnostic results from the physicians.

### SUMMARY OF THE INVENTION

An object of the present invention is to solve the aforementioned problems and provide an inexpensive, extremely safe genetic diagnosis/analysis apparatus and a genetic diagnosis support system that expedite completion of testing, eliminate the need for patients to revisit the hospital, provide a broad range of tests, and facilitate retesting.

Another object of the present invention is to provide a system that does not have any components for inferring diseases and can provide extremely precise answers to questions regarding the reliability and reproducibility of diagnostic results.

### BRIEF DESCRIPTION OF THE DRAWINGS

FIG. 1 is a schematic view showing one embodiment of the genetic diagnosis support system and the main components thereof according to the present invention.

FIG. 2 is a block diagram showing one embodiment of the genetic diagnosis/analysis apparatus.

FIG. 3 is a schematic view showing the structure of a network-connected content server.

FIG. 4 is a schematic view showing another embodiment and the main components thereof according to the present invention.

### DETAILED DESCRIPTION OF THE PREFERRED EMBODIMENTS

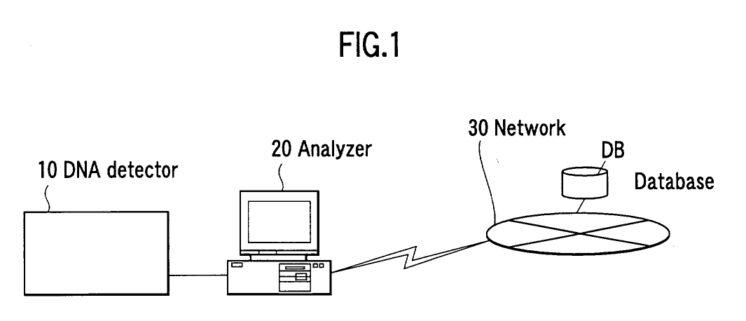

Preferred embodiments of the present invention will be described in detail herein with reference to the accompanying drawings. FIG. 1 is a schematic view showing one embodiment of the genetic diagnosis support system according to the present invention. In FIG. 1, numeral 10 denotes a DNA detector for detecting gene expression, gene representation, or the single nucleotide polymorphisms (SNPs) of genes from samples of blood, bodily fluids, or affected parts (hereinafter generically referred to as samples) collected from patients.

Numeral 20 denotes an analyzer that refers to a diagnosis protocol (contents) database, which analyzes the results of detection by aforementioned DNA detector 10, and outputs information (correlation data) regarding the types, progress, stages and prognoses of diseases, drug sensitivities and resistance, metabolic capability and other items. In most cases, a computer is used as the analyzer.

In correspondence with gene expression, the amount of gene expression, or the SNPs(here, also simply referred to as a sequence) of genes, the diagnosis protocol database stores a variety of information, such as the type of disease indicated by the aforementioned gene-related matter, the progress, stages and prognoses of diseases, or the diathesis, drug sensitivity, drug resistance, and metabolic capability of the patient in question.

It should be noted here that the diagnosis protocol database to be referred to by the analyzer can be either the diagnosis protocol database stored in the network-connected content server that is discussed hereinafter, or the diagnosis protocol database that has been previously incorporated into the analyzer by reading only the relevant data of the former database on the content server.

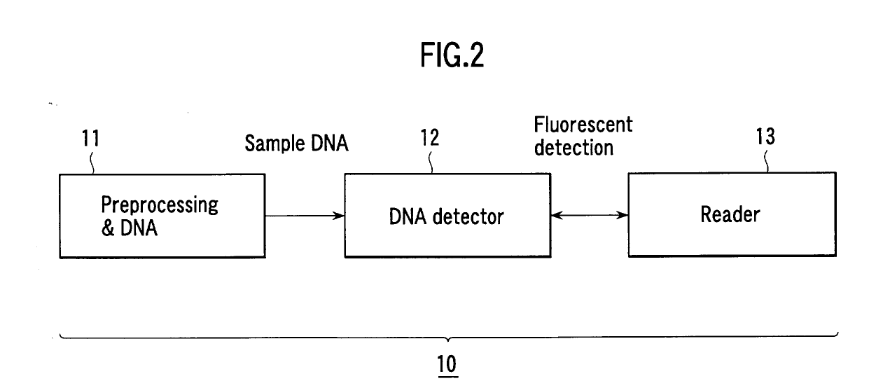

FIG. 2 is a block diagram showing the details of DNA detector 10. In the figure, numeral 11 denotes a preprocessing unit responsible for preprocessing, such as extracting target DNA from collected samples. Other processes, such as attaching a fluorescent marker element to target DNA or performing DNA amplification using a PCR method, are also carried out by this unit.

Numeral 12 denotes a DNA detector that hybridizes probe DNA with target DNA using DNA chips, in order to detect DNA. Numeral 13 denotes a reader that reads, by means of fluorescence, the expression, gene representation, or the sequence of, the target DNA that combines with the probe DNA.

It should be noted that the system comprising DNA detection means 10 for detecting gene expression or sequence, and analyzer 20 is referred to here as a genetic diagnosis/analysis apparatus. As DNA detection means 10, it is possible to use the biochip described by the inventor of the present invention in the Japanese Laid-open Patent Application 2001-235468 or Patent Application 2001-1176712.

Numeral 30 denotes a network to which analyzer 20 is connected, and is also a local area network (LAN) in a company, institution, hospital or university, or a wide-area network. A content server or servers in which a diagnosis protocol database or the like is provided is connected to such a network as mentioned above.

Note that a plurality of genetic diagnosis/analysis apparatus can be connected to network 30, from each of which it is possible to refer to diagnostic protocols in a content server, as necessary.

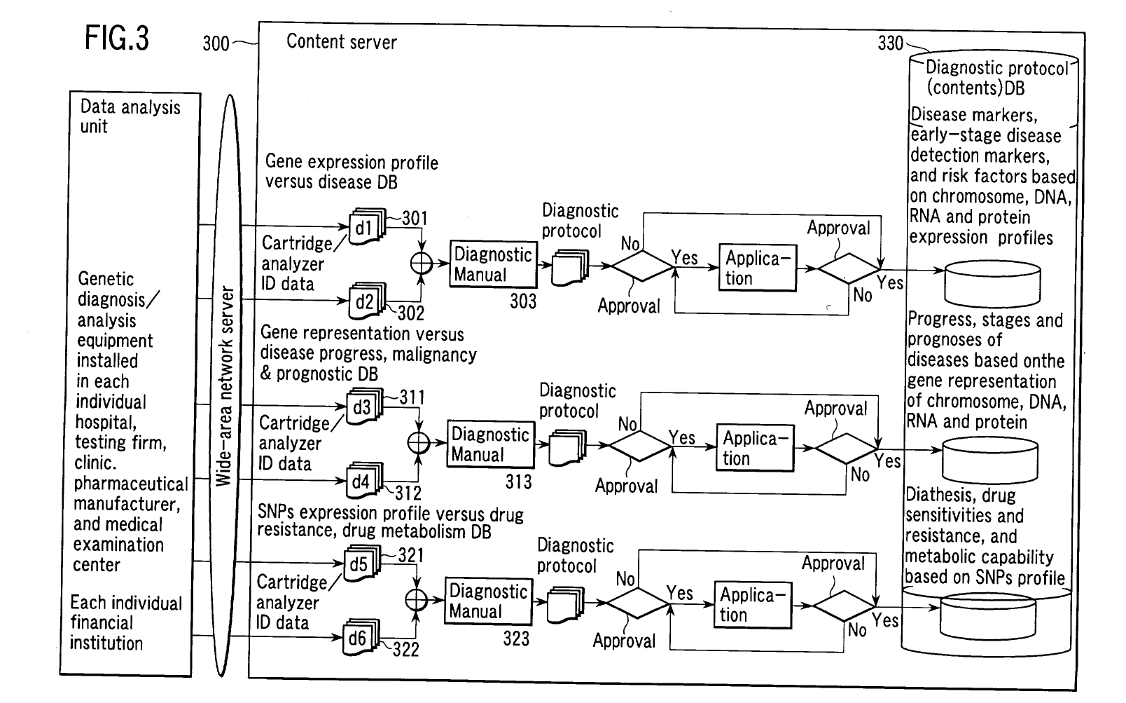

FIG. 3 is a schematic view showing the conceptual structure of a content server. In this server, correlation data 301 correlating expression profiles of chromosomes, DNA, RNA and protein with diseases (for example, infectious diseases, cancer, and lifestyle-related diseases), correlation data 311 correlating amounts of chromosomes, DNA, RNA and protein expression with the progress, stages or prognoses of diseases, and correlation data 321 correlating expression profiles of SNPs with diathesis, drug sensitivity, drug resistance and metabolic capability, can be combined as necessary with their respective diagnostic expertise 302, 312 and 322, in order to create diagnostic manuals 303, 313 and 323.

These items of correlation data or diagnostic expertise are input through network 30 from each genetic diagnosis/analysis apparatus in a university, research laboratory, hospital or company.

In content server 300, diagnostic protocols are determined according to the created diagnostic manuals and accumulated in diagnosis protocol database 330. If approval of a government or public office is required for these diagnostic protocols, the protocols are registered with the database after an application is made and such approval is gained.

The diagnosis protocol database is updated in real time as necessary, as it is supplied with information from a genetic diagnosis/analysis apparatus. Consequently, it is possible for each university, research laboratory, hospital and company to have access to the database and to easily refer to the latest diagnostic content.

The operation of a system configured in such a manner as discussed above is described herein. Samples are collected from a patient according to the diagnosis of a physician and processed by preprocessing unit 11 to extract DNA.

The DNA thus extracted is mixed into a solution, and the solution is flowed onto a DNA chip placed on DNA detector 12, in which the DNA is hybridized with DNA probes.

After hybridization, unnecessary DNA targets are removed and the expression, gene representation, or the sequence of-target DNA that has combined with the fluorescent tag is read with reader 13.

The information thus read is then input to analyzer 20. Analyzer 20 analyzes the information according to diagnostic protocols previously stored therein, or according to diagnostic protocols in the database of content server 300 on a network. Thus, the analyzer obtains information on diseases (for example, infectious diseases, cancer, and lifestyle-related diseases) or on diathesis, drug sensitivity, drug resistance and metabolic capability from the expression of DNA, and information on the progress, stages, and prognoses of diseases from gene representation.

The physician pronounces a diagnosis according to the information thus gained, and concludes the type of disease, a treatment plan, and preventive actions.

The advantages of using such a genetic diagnosis support system as described above are as follows:

(1) A series of processes, from sample collection to analysis based on diagnostic protocols, is carried out without interruption and completed in a short period of time (approximately one hour under normal conditions).

(2) Unlike conventional methods of diagnosis in which a surmised disease is identified from indirect phenomena, such as blood pressure or urine, the aforementioned system allows a physician to examine genes and directly confirm the type and state of the disease, thus reducing the possibility of misdiagnosis.

(3) Reportedly, 40% of prescription medicine has no effect on patients and about one hundred thousand people die annually from side effects in the United States alone. Examining genes makes it possible, however, to know the type of medicine agreeable to the diathesis of patients, thus reducing the possibility of suffering from side effects.

(4) Conventional methods of diagnosis in which the possible existence of a disease is determined from indirect phenomena, such as blood pressure or urine, do not provide a means of discovering diseases early. According to the present invention, however, genes are examined and therefore the type and progress of diseases can be made known at an earlier stage thereof, thus facilitating expedient provision of treatment.

(5) The system is not susceptible to virus contamination and is superior in safety, since samples are not removed from their original containers or equipment in any of the steps, from blood collection to analysis and disposal.

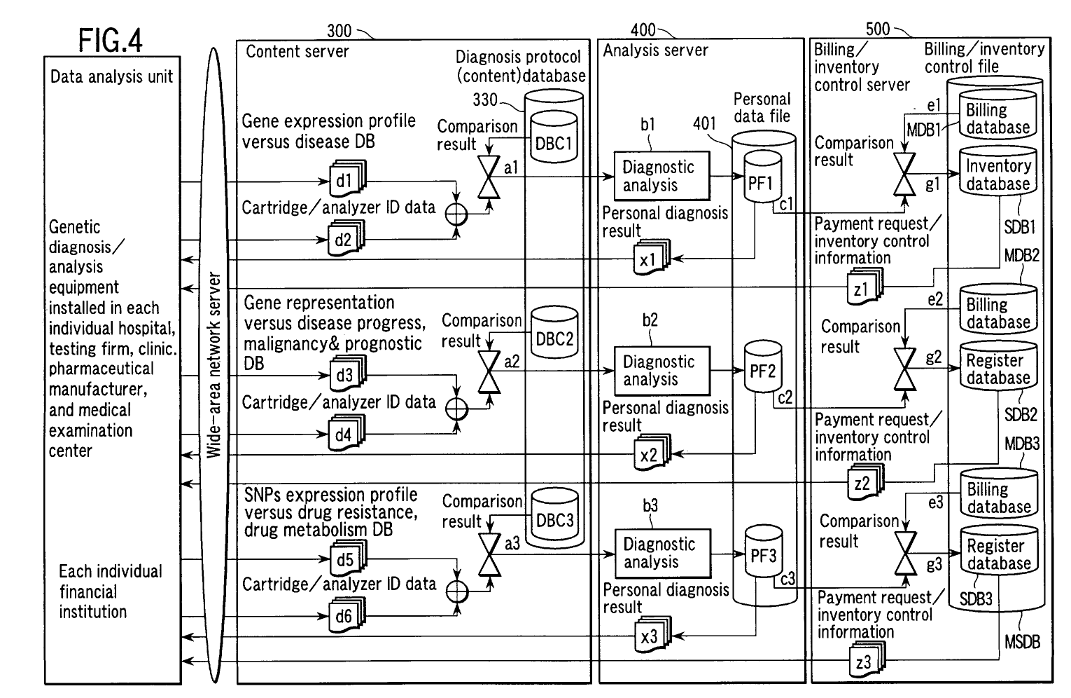

FIG. 4 is a schematic view showing another embodiment of the present invention.

From a genetic diagnosis/analysis apparatus in individual universities, research laboratories, hospitals or companies, expression profile analysis data or gene representation analysis data and the ID data of cartridges or analysis equipment used when acquiring such analysis data are sent to content server 300. In content server 300, these input data items are compared with a diagnosis protocol database.

In addition to content server 300, analysis server 400 and billing/inventory control server 500 are connected to the network. Analysis server 400 performs diagnostic analysis according to the results of comparisons made by the content server, and stores diagnosis results in personal data file 401.

In billing server 500, information regarding treatment, dosage and other actions taken for the patient in question is extracted from personal information files in analysis server 400 and compared with a billing database in billing/inventory control file 501. Then, expenses involved in diagnostic analysis and dosage, for example, are determined and this information is stored in a register database.

The contents of the register database can be read from each genetic diagnosis/analysis apparatus.

Using such a genetic diagnosis support system as described above in which a genetic diagnosis/analysis apparatus, content server 300, analysis server 400 and billing server 500 are systematically connected to one another through a network, it is possible to immediately obtain a total sum of expenses involved in, for example, diagnostic analysis from the billing server, by sending profile analysis data on the expression (or the amount of expression) of biopolymer from the genetic diagnosis/analysis apparatus in individual universities, research laboratories, hospitals or companies and the ID data of cartridges or analysis equipment to content server 300. Each university, research laboratory, hospital or company need not calculate expenses using its own system each time a diagnosis is made.

It is to be understood that the present invention is not restricted to the foregoing embodiments; rather, many other alterations and modifications thereof may be made without departing from the spirit and essential characteristics thereof. It is therefore intended that such alterations and modifications be covered by the appended claims.

For example, DNA chips are not limited to one type only but different types may be used as applicable. In other words, it is possible to use dedicated DNA chips as necessary, such as those dedicated to lung cancer or type C hepatitis.

It is also possible to amplify target DNA in a preprocessing stage by using gene amplification means, such as PCR, if the amount of the available sample is limited.

As described heretofore, the following advantages are inherent in the present invention.

(1) A series of processes, from sample collection to analysis based on diagnostic protocols, is carried out without interruption and completed in a short period of time (approximately one hour under normal conditions).

(2) A physician can examine genes and directly confirm the type and state thereof, thus preventing misdiagnosis.

(3) Since genes are examined, it is possible to determine the type of medicine and treatment method agreeable to the diathesis of patients, thus eliminating the side effects of the medicine.

(4) Since genes are examined, it is possible to know the type and progress of diseases at an earlier stage thereof, thus allowing early treatment to be provided.

(5) The system is not susceptible to virus contamination and is superior in safety, since samples are not removed from their original containers or equipment in any of the steps, from blood collection to analysis and disposal.

(6) Since a variety of DNA chips are available, it is possible to perform detailed examinations using a dedicated DNA chip according to the sample under test.

(7) Re-examination can be performed easily.

What is claimed is:

1. A genetic diagnosis/analysis apparatus using detection chips for detecting gene expression, gene representation, or the SNPs of genes, from samples of blood, bodily fluids, or affected parts collected from patients, comprising:
gene detection means for preprocessing samples and detecting gene expression, gene representation, or the SNPs of genes; and
an analyzer for analyzing the results of detection by said gene detection means by referring to diagnostic protocols and for obtaining information on the disease in question and other related information,
wherein the preprocessing of said samples and gene detection are performed within the same container and the analysis of the resulting data is performed without interruption and completed within an hour.

2. The genetic diagnosis/analysis apparatus of claim 1, wherein said diagnostic protocols correlate the gene expression profiles, gene representation profiles, or the SNP profiles with at least one item among the types, progress, stages and prognoses of diseases, drug sensitivities and resistance, and metabolic capability, and serve as diagnostic criteria.

3. The genetic diagnosis/analysis apparatus of claim 1 or 2, wherein said preprocessing unit, said gene detection means and said analyzer are integrated into a single system.

4. The genetic diagnosis/analysis apparatus of claim 1 or 3, wherein a variety of dedicated chips can be used as said detection chips according to the purpose of the examination.

5. The genetic diagnosis/analysis apparatus of claim 1, 2 or 3, wherein gene amplification means, such as PCR, is used.

6. The genetic diagnosis/analysis apparatus of any of claims 1 to 5, wherein DNA detection and analysis is performed a series of steps, from blood collection to analysis without any disposal.

7. A genetic diagnosis support system, wherein a genetic diagnosis/analysis apparatus using detection chips for detecting gene expression, gene representation, or the SNPs of genes, from samples of blood, bodily fluids, or affected parts collected from patients, comprising gene detection means for preprocessing samples and detecting gene expression, gene representation, or the SNPs of genes and an analyzer for analyzing the results of detection by said gene detection means by referring to diagnostic protocols and for obtaining information on the disease in question and other related information, wherein the preprocessing of said samples and gene detection are performed within the same container and the analysis of resulting data is performed without interruption and completed within an hour, and a content server having a database of diagnostic protocols correlating gene expression or gene sequence profiles with at least one item among the types, progress, stages and prognoses of diseases, drug sensitivities and resistance, and metabolic capability, and serving as diagnostic criteria, are connected to a network, wherein diagnosis can be performed by sending the analysis data of test results from said genetic diagnosis/analysis apparatus to said content server and reading corresponding diagnostic protocols.

8. The genetic diagnosis support system of claim 7, wherein an analysis server for comparing the gene expression or gene representation or the like, sent from said genetic diagnosis/analysis apparatus with diagnostic protocols saved in a content server to perform diagnostic analysis, and saving the results of diagnostic analysis in an information file, and a billing server for comparing data in the information file of said analysis server with data in a billing database to determine expenses involved in diagnosis and analysis are connected to said network, so that expenses involved in diagnosis and analysis performed by said genetic diagnosis/analysis apparatus can be determined through said network.

9. The genetic diagnosis support system of claim 7 or 8, wherein a variety of dedicated chips can be used as said detection chips according to the purpose of examination.

10. The genetic diagnosis support system of claim 7, 8 or 9, wherein said genetic diagnosis/analysis apparatus can detect genes by means of fluorescent detection.

11. The genetic diagnosis support system of claim 7, 8 or 9, wherein a gene amplification means, such as PCR, is used.

12. The genetic diagnosis support system of any of claims 7 to 11, wherein DNA detection and analysis is performed without removing samples from the original containers thereof throughout a series of steps, from blood collection to analysis and disposal.

13. The genetic diagnosis support system of any of claims 7 to 12, wherein the diagnosis protocol database of said content server can be updated at any time from said genetic diagnosis/analysis apparatus.
